(19) United States
(12) Patent Application Publication
(10) Pub. No.: US 2004/0014220 A1
(43) Pub. Date: Jan. 22, 2004

(54) Method for introducing nucleic acids and other biologically active molecules into the nucleus of higher eukaryotic cells by means of an electrical current

(76) Inventors: Gregor Siebenkotten, Strasse (DE); Rainer Christine, Koln (DE); Ludger Altrogge, Pulheim (DE); Marion Gremse, Koln (DE); Dietmar Lenz, Koln (DE); Sabine Poppenborg, Steinhagen (DE); Gudula Riemen, Langenfeld (DE); Kirsten Rothmann, Berlin (DE); Corinna Thiel, Koln (DE)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/312,244

(22) PCT Filed: Jun. 27, 2001
(86) PCT No.: PCT/EP01/07348

(30) Foreign Application Priority Data
Jun. 27, 2000 (DE) 100 31 179.2

(51) Int. Cl. C12N 15/87; C07H 21/04; C07K 14/00

(52) U.S. Cl. 435/461; 536/23.1; 530/395

(57) ABSTRACT

The invention relates to a novel method allowing the transport of DNA and/or other biologically active molecules into the nucleus of eukaryotic cells using electrical current, independently from cell division and with low cell mortality.

The invention relates to a novel method which allows the transport of DNA and/or other biologically active molecules into the nucleus of higher eukaryotic cells using electric current, independently of cell division and with low cell mortality. The invention further relates to a method which reduces the time between transfection and cell analysis, and thus greatly accelerates the experiments. Optimized electrical pulses are described, which may be used for the nuclear localization of DNA and/or other biologically active molecules.

### BACKGROUND OF THE INVENTION

Since the nucleus is the functional location of eukaryotic DNA, external DNA has to enter the nucleus in order to be transcribed. Conventional transfection methods only cause transport of DNA through the cell membrane into the cytoplasm. Only because the nuclear envelope is temporarily disintegrated during cell division of higher eukaryotes, can the DNA enter the nucleus passively, so that its encoded proteins can be expressed. Only very small DNA molecules (oligonucleotides) are able to diffuse freely through pores in the nuclear envelope. For the efficient transfection of resting cells or of cells with low rates of division conditions have to be provided which result in larger DNA molecules being able to enter the nucleus in sufficient quantities through the closed nuclear membrane. The method described here allows this in higher eukaryotic cells.

### PRIOR ART

It has long been known that DNA can be introduced from a buffer into cells with the help of electric current. However, the experimental conditions described earlier are limited to the transport of DNA into the cytoplasm of higher eukaryotic cells, so that the expression of transfected DNA remains dependent on the disintegration of the nuclear envelope during cell division. None of the known methods addresses the electrically targeted introduction of DNA into the nucleus of higher eukaryotic cells. A system which is optimized for electrical nuclear transport is not yet known.

The development of electroporation is based on the observation that biological membranes temporarily become more permeable through the effect of short electrical pulses (Neumann & Rosenheck 1972). In 1976 Auer et al. described the uptake of DNA in red blood cells through electric current.

The first report of electroporation of cell line cells dates from 1982 (Neumann et al.). A murine fibroblast cell line was transfected using short pulses having a field strength of 8 kV/cm and a duration of 5 μs each, mostly in a series of three pulses at intervals of three seconds. Two weeks later, an analysis was conducted. No electrical nuclear transport was observed.

By shorting of a transformer, Potter et al. (1984) also generated a field strength of 8 kV/cm and used it for transfection of cell line cells. However, the current was limited to a maximum of 0.9 A. Again, no electrical nuclear transport was observed.

In the course of the development, increasingly longer discharges with lower voltages were used since a field strength of approx. 1 kV/cm appeared to be sufficient for an optimal opening of the pores in the cell membrane (with an average cell diameter of 10-20 μm). Thus, most of the commercial devices for electroporation of higher eukaryotic cells and the supplied protocols are optimized for transfection with these field strengths.

In one case (Bertling et al., 1987), the kinetics of the distribution of the DNA in cytoplasm and nucleus was followed in a dividing cell line. Apart from the increase in the DNA concentration, no other attempt was made to optimize the early uptake of DNA into the nucleus. More specifically, it was not investigated if electrical parameters could have an influence on the distribution.

Since 1986 patents have been applied for which relate to electroporation as a method of transfection. Mainly they describe device constructions and pulse forms. None addresses the problem of non-mitotic transport of DNA into the nucleus.

U.S. Pat. No. 4,750,100 to Bio-Rad Laboratories, Richmond, USA (1986) describes a specific device construction providing a maximum of 3000 V at a maximum of 125 A by condenser discharge.

U.S. Pat. No. 5,869,326 (Genetronics, Inc., San Diego, USA, 1996) describes a specific device construction, with which two, three, or more pulses may be generated using two separate power sources. However, U.S. Pat. No. 5,869,326 does not show that these pulses have an effect beyond the transport of DNA into the cytoplasm.

U.S. Pat. No. 6,008,038 and EP 0 866 123 A1 (Eppendorf-Netheler-Hinz GmbH, Hamburg, 1998) describe a device, with which short pulses of 10-500 μs and max. 1.5 kV may be generated, but again do not indicate that certain conditions may result in the transport of DNA into the nucleus.

The methods known at present do not allow the efficient transport of DNA and/or other biological molecules into the nucleus with low cell mortality.

It is therefore an object of the invention to provide a method which allows the efficient transport of DNA and/or other biological molecules into the nucleus with low cell mortality.

It is a further object of the invention to provide a method of greatly reducing the time between transfection and subsequent analysis of transfected cells.

The objects are solved by the subject-matter of the patent claims.

In the present application, specific electrical conditions which mediate the efficient nuclear transport of DNA, and discharges and currents resulting in a particularly low cell mortality are described for the first time. Buffers optimized for low cell mortality, and experimental procedures are described as well.

### DESCRIPTION OF THE INVENTION

In the method described here, very high field strengths of 2 to 10 kV/cm are used to aid DNA and/or other biologically active molecules in entering the nucleus independently of cell division. These field strengths are substantially higher than the ones generally used in electroporation, and are also higher than the field strengths that are sufficient for efficient opening of cell membrane pores (1 kV/cm in average, according to Lurquin, 1997).

Therefore, the subject of the invention is a method for introducing biologically active molecules into the nucleus of eukaryotic cells using electric current, the introduction into the nucleus being achieved by a pulse having a field strength of 2-10 kV/cm, and a duration of at least 10 μs, and a current of at least 1 A. The high voltages used may result in the generation of pores in both membranes of the nuclear envelope, or the nuclear pore complexes may become more permeable for molecules, thus enabling very efficient transport of the biologically active molecules into the nucleus. The pulse is required to have a duration of at least 10 μs to achieve a nuclear transport effect.

The term “biologically active molecule”, as used herein, comprises nucleic acids, peptides, proteins, polysaccharides, lipids, or combinations thereof, provided they demonstrate biological activity in the cell.

The introduction of nucleic acids, peptides, proteins, and/or other biologically active molecules in the nucleus may preferably be achieved by a pulse having a field strength of 3-8 kV/cm, the duration of the pulse not exceeding 200 μs in a preferred embodiment of the invention. A voltage of 1-2 V over a cell results in an efficient and reversible opening of the pores in the cell membrane (Zimmermann et al., 1981). This corresponds to 1 kV/cm in average at a cellular diameter of 10-20 μm. A distinctively higher voltage should result in irreversible membrane collapse, even at pulse durations of less than 1 ms (Zimmermann et al., 1981). However, this does not occur using the method according to the present invention. In the method according to the present invention it is especially preferred to keep the pulse duration at a maximum of 200 μs, which is short enough, that even at 2-10 kV/cm, preferably 3-8 kV/cm, no substantial irreversible membrane damage may occur, but at the same time long enough still to achieve a nuclear transport effect.

In a preferred embodiment of the method, the pulse is followed without interruption by a current flow of 1 A to maximally 2.5 A with a duration of 1 ms to max. 50 ms.

The transfection using electric current is based on two effects: electroporation of the cell membrane and electrophoresis of DNA through the resulting membrane pores. The described electroporation pulses comply with both conditions independently of their generation and their form in such a way, that a voltage is present at the beginning of the pulses, which is sufficient to open the pores in the cell membrane of the respective cell type, and that the further course of the pulse is sufficient for DNA electrophoresis.

In a preferred embodiment of the present invention, field strengths of 2-10 kV/cm are used for 10-200 μs at the beginning of the pulse for the transport of DNA and/or other biologically active molecules through the cell membrane into the nucleus, the subsequent electrophoresis taking place under conventional conditions.

Through the short duration of the strong pulse and the low strength and/or short duration of the subsequent current flow, the transfection with electrical nuclear transport is optimized for a high survival rate of the cells despite the very high initial voltages. At the same time, a fine tuning can be performed depending on the type of primary cells. As the short and very high current pulse may contribute to the electrophoresis of DNA, it may also allow that the subsequent current flow may be strongly reduced or completely omitted for a few cell types

In a preferred version, a cuvette filled with buffer, cells and nucleic acids (and possibly other biologically active molecules) is exposed to a short impulse (with a length of 10-200 μs) with a field strength of 2-10 kV/cm, followed by a current of max. 2.5 A for up to 50 ms.

In a more preferred version, a cuvette filled with buffer, cells and nucleic acids (and possibly other biologically active molecules) is exposed to a short impulse (with a length of 10-100 μs) with a field strength of 3-8 kV/cm, followed by a current of max. 2,2 A for up to 30 ms.

As this transfection method is independent of cell division, dividing cells as well as resting or primary cells with low division activity may be transfected.

In an embodiment of the invention, primary cells, such as peripheral human blood cells, preferably being T cells, B cells, or pluripotent precursor cells of human blood, are transfected.

In another preferred embodiment, the eukaryotic cells comprise embryonic cells of neurons from humans, rats, mice or chicken.

Another preferred embodiment comprises human bone marrow cells.

The eukaryotic cells transfected with the method according to the invention may be used for diagnostic methods, and for preparing a drug for ex vivo gene therapy.

The transfection efficiency may be increased with dividing primary cells and cell lines, since the DNA does not need to stay in the cytoplasm until cell division, where it may be degraded, and since the cells that have not undergone cell division at the time of the analysis may be analyzed as well.

Furthermore, an analysis is possible shortly after the transfection already, resulting in a significant acceleration of the experiments. In transfection experiments with expression vectors, an analysis may be performed as soon as approx. 20 hours after the transfection, depending on the promotor and the expressed protein. Due to the short stay of the transfected DNA in the cytoplasm, the DNA will hardly be exposed to the effect of nucleases.

With electrical nuclear transport, higher amounts of DNA may be transported into the nucleus of dividing cells than may be expected due to cell division alone. Both substantially increase the likelihood of integration of complete expression cassettes.

The term “electrical nuclear transport” describes the transport of biologically active molecules into the nucleus of higher eukaryotic cells, which is caused independently of cell division and by electric current.

Preferably, the biologically active molecule, that is meant to enter the nucleus, comprises a nucleic acid, particularly DNA, or includes at least one nucleic acid portion.

The nucleic acids may be present in a complex or in association with peptides, proteins, polysaccharides, lipids or combinations or derivatives of these molecules. The molecules which are complexed or associated with the nucleic acids aid the integration of the transferred nucleic acid into the genome of the cell, the intranuclear localization or retention, the association with the chromatin, or the regulation of expression.

In a preferred embodiment, the molecules complexed with the nucleic acid and being used for the integration of the transferred nucleic acid into the genome of the cell are selected from the group comprising retroviral integrases, prokaryotic transposases, eukaryotic transposases, sequence specific recombinases, topoisomerases, E. coli recA, E. coli recE, E. coli recT, phage λ red α, phage λ red β and phage λ terminase.

In a particularly preferred embodiment, the molecules complexed or associated with the nucleic acid and being used for the intranuclear retention or the association with the chromatin comprise domains of the EBV protein EBNA-1. These domains include aminoacids 8-54 and/or 72-84, or 70-89, and/or 328-365 of the EBNA-1 protein (Marechal et al., 1999).

A buffer suitable for the use in the method according to the invention is “buffer 1” having the following composition: 0.42 mM Ca(NO₃)₂; 5.36 mM KCl; 0.41 mM MgSO₄; 103 mM NaCl; 23.8 mM NaHCO₃; 5.64 mM Na₂HPO₄; 11.1 mM d(+)− glucose; 3.25 μM glutathione; 20 mM Hepes; pH 7.3.

For introduction of nucleic acids into the nucleus of eukaryotic cells, the following protocol may be carried out: 1×10⁵−1×10⁷ cells and up to 10 μg DNA are incubated in 100 μl buffer 1 in a cuvette with 2 mm electrode spacing for 10 min at room temperature, and are then transfected according to conditions according to the invention. Immediately afterwards, the cells are rinsed out of the cuvette with 400 μl cell culture medium without serum, and are incubated for 10 min at 37° C. Then, the cells are plated in cell culture medium (with serum) with a temperature of 37° C.

For example, electrical nuclear transport of proteins may take place according to the following protocol: Up to 10 μg protein in 100 μl suitable buffer are transfected into 1×10⁵1×10⁷ cells according to the conditions of the invention. Immediately afterwards, the cells are rinsed out of the cuvette with 400 μl cell culture medium without serum, and incubated for 10 min at 37° C. Then, the cells are plated in cell culture medium (with serum) with a temperature of 37° C., and are analyzed after an incubation period of up to 6 h.

Suitable cuvettes are for example those with an electrode spacing of 2 mm or 1 mm, such as commercially available cuvettes for the electroporation of prokaryotes.

The Used Abbreviations have the Following Meaning According to the Invention:

Apart from the abbreviations listed in the Duden dictionary, the following abbreviations were used:

| | |
|---|---|
| FACS | flurorescence activated cell sorting |
| FCS | fetal calf serum |
| H | hour |
| KV | kilovolts |
| Ms | millisecond |
| μs | microsecond |
| PBMC | peripheral mononuclear blood cells |
| PE | phycoerythrin |

### FIGURES

The invention is described further by the following figures:

FIGS. 1(a) and 1(b) show the transfection efficiency of T helper cells relative to the field strength at a pulse with a duration of 40 μs (a), and in relation to the pulse duration at 5 kV/cm (b).

FIGS. 2 (a) and (b) show the transfection efficiency of T helper cells after a pulse of 5 kV/cm for 40 μs, followed without a interruption by a current flow of different strengths and durations.

FIG. 3 shows the FACScan analysis of PBMC transfected with the H-2K^{k} expression vector. The cells were subsequently incubated with the digoxigenin-coupled anti-H-2K^{k} antibody and then with the Cy5-coupled anti-digoxigenin antibody, as well as with a phycoerythrin (PE)-coupled anti-CD4 antibody for identification of the T helper cells, and were analyzed by flow cytometry. The number of dead cells was determined by propidium iodide staining (unstained fluorescence channel FL3) (SSC=side scatter; FSC=forward scatter).

FIG. 4 is a FACScan analysis of the electrical nuclear transport in primary (dividing) endothelial cells from human umbilical cord (HUVEC), transfected by a 70 μs pulse of 5 kV/cm, followed by a current flow of 2.2 A for 10 ms. The cells were subsequently incubated with the digoxigenin-coupled anti-H-2K^{k} antibody and then with the Cy5-coupled anti-digoxigenin antibody, as well as with propidium iodide (unstained fluorescence channels FL2 and FL3), and were analyzed by flow cytometry (FACScan) (SSC=side scatter; FSC=forward scatter).

FIG. 5 is a FACScan analysis of the electrical nuclear transport in a cell line (HeLa) 3 hours after it was transfected by a 100 μs pulse of 4 kV/cm with an H-2K^{k} expression vector. After 4 hours, the cells were subsequently incubated with the digoxigenin-coupled anti-H-2K^{k} antibody and then with the Cy5-coupled anti-digoxigenin antibody, as well as with propidium iodide (unstained fluorescence channels FL2 and FL3), and were analyzed by flow cytometry (FACScan) (SSC=side scatter; FSC=forward scatter).

FIG. 6 shows the electrical nuclear transport of a transcription activator protein (HPV 18-E2) by means of the analysis of its effect on a reporter construct (pC18Sp1luc) in HeLa cells. The measurement was performed 6 hours after protein and plasmid were introduced into the cells by a 100 μs pulse of 4 kV/cm.

FIG. 7 shows two diagrams of flow cytometric measurements of the electrical nuclear transport of DNA-lac-repressor complexes into CHO cells 5½ hours after they were transfected with the DNA-protein complex by a 70 μs pulse of 5 kV/cm followed without interruption by a current flow of 2.2 A and 60 ms.

FIG. 8 shows two diagrams of flow cytometric measurements of the electrical nuclear transport of peptide-DNA complexes into CHO and K562 cells. The complexes were introduced into the CHO cells by a pulse of 5 kV/cm for 70 μs, followed by a current flow of 2.2 A for 40 ms, and into the K562 cells by a pulse of 5 kV/cm for 100 μs, followed by a current flow of 5 A for 10 ms. The analysis was performed four hours after the transfection.

### EXAMPLES

The following examples illustrate the invention, but are not to be conceived as to be limiting.

### Example 1

Electrical Nuclear Transport in Relation to the Field Strength and the Pulse Duration

Freshly prepared unstimulated (non dividing) mononuclear cells from peripheral human blood (PBMC) were transfected with a vector coding for the heavy chain of the murine MHC class I protein H-2K^{k}. 1×10⁶ cells together with 10 μg vector DNA in buffer 1 were transferred at room temperature into a cuvette with 2 mm electrode spacing, and were transfected under the described conditions. Immediately afterwards, the cells were rinsed out of the cuvette with 500 μl RPMI medium (without fetal calf serum, FCS), incubated for 10 min at 37° C, and were then transferred to a culture dish with prewarmed medium (with FCS). After 5 h incubation, the cells were subsequently incubated with the digoxigenin-coupled anti-H-2K^{k} antibody and then with the Cy5-coupled anti-digoxygenin antibody, as well as with a phycoerythrin (PE)-coupled anti-CD4 antibody for identification of the T helper cells, and were analyzed by flow cytometry (FACScan). The number of dead cells was determined by propidium iodide staining.

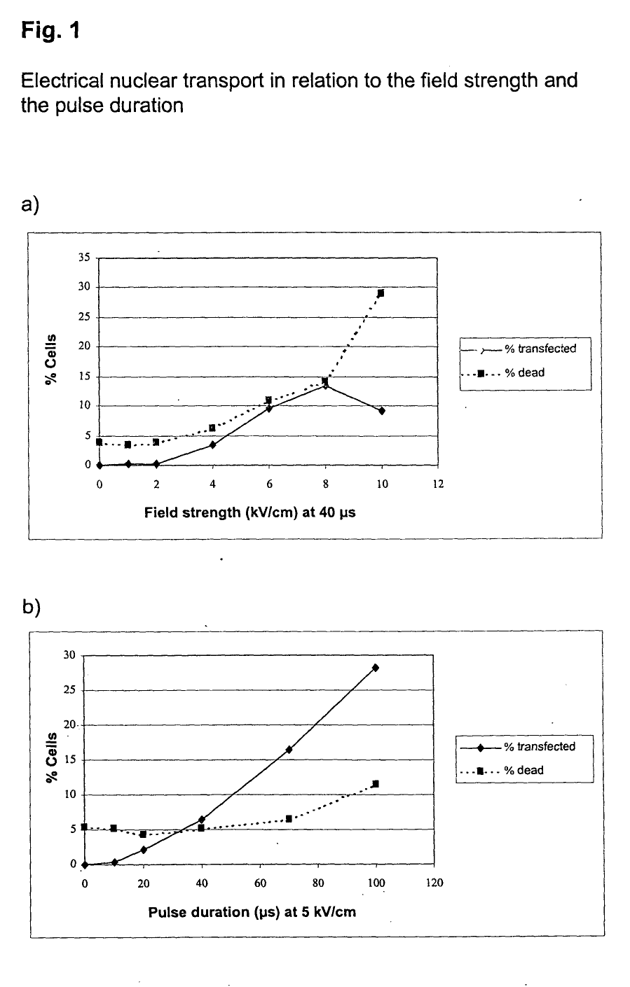

FIG. 1 shows the transfection efficiency of T helper cells in relation to the field strength at a pulse duration of 40 μs (a), and in relation to the pulse duration at 5 kV/cm (b).

### Example 2

Increase of the Efficiency of the Electrical Nuclear Transport by a Current Flow Following the Pulse

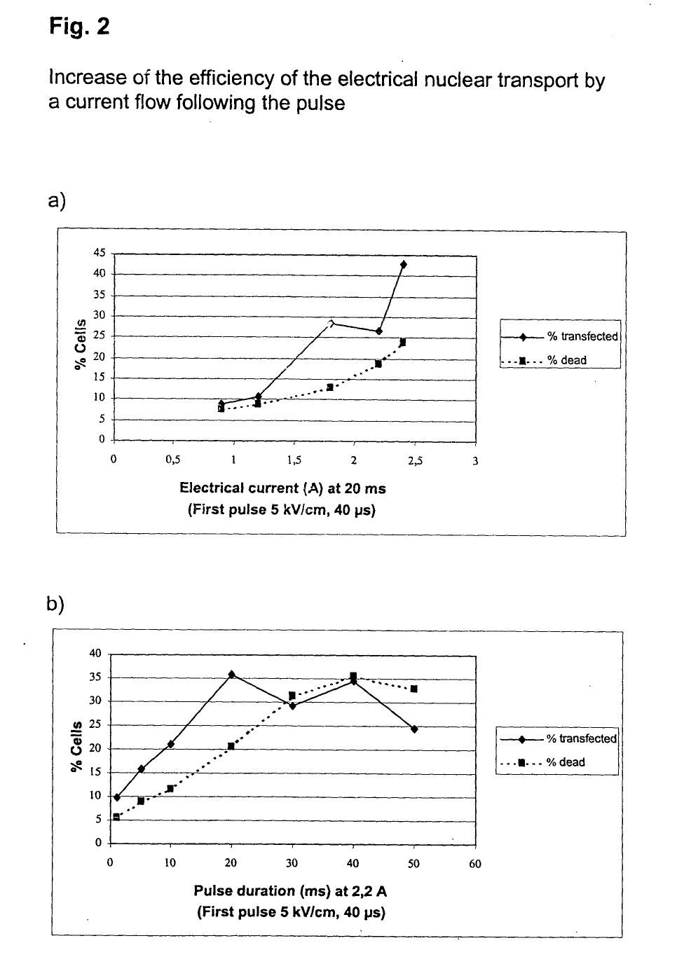

Freshly prepared unstimulated PBMC were transfected with an H-2K^{k} expression vector as described in example 1. A pulse of 5 kV/cm for 40 μs was followed without interruption by a current flow of different strengths and duration. After 5 h incubation the cells were analyzed as in example 1, and the transfection efficiency of T helper cells was determined (FIG. 2).

### Example 3

Transfection of PBMC

Freshly prepared unstimulated PBMC were, as described in example 1, transfected with an H-2K^{k} expression vector by a 40 μs pulse of 5kV/cm, followed by a current flow of 2.2 A for 20 ms, and were analyzed as in example 1.

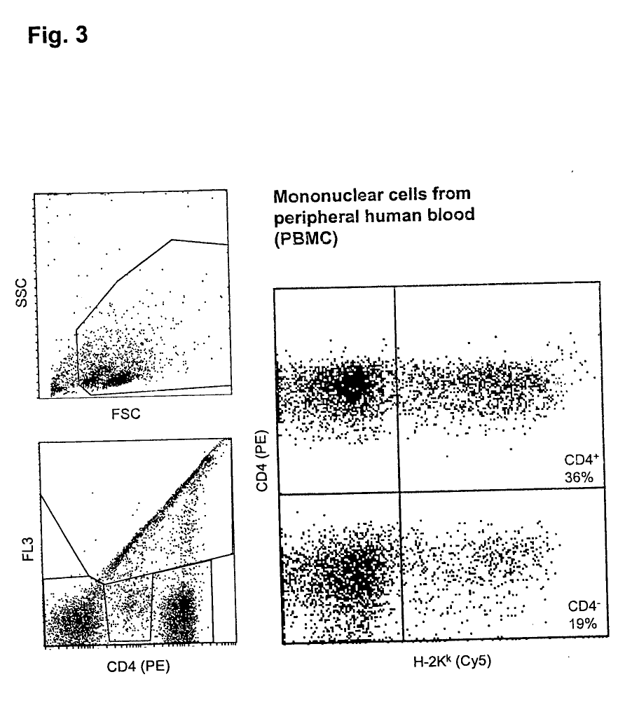

FIG. 3 shows the analysis of the portion of transfected cells in the CD4-positive and the CD4-negative fractions of the PBMC. 36% of the CD4⁺ cells and 19% of the CD4⁻ cells express the transfected DNA. Three fourths of the mortality rate of 26% are due to the transfection procedure.

### Example 4

Electrical Nuclear Transport in Primary (Dividing) Endothelial Cells from Human Umbilical Cord (HUVEC)

As described in example 1, HUVEC were transfected with an H-2K^{k} expression vector by a 70 μs pulse of 5 kV/cm, followed by a current flow of 2.2 A for 10 ms, and after 4 hours subsequently incubated with digoxigenin-coupled anti-H-2K^{k} antibody and then with the Cy5-coupled anti-digoxigenin antibody, as well as with propidium iodide, and were analyzed by flow cytometry (FACScan).

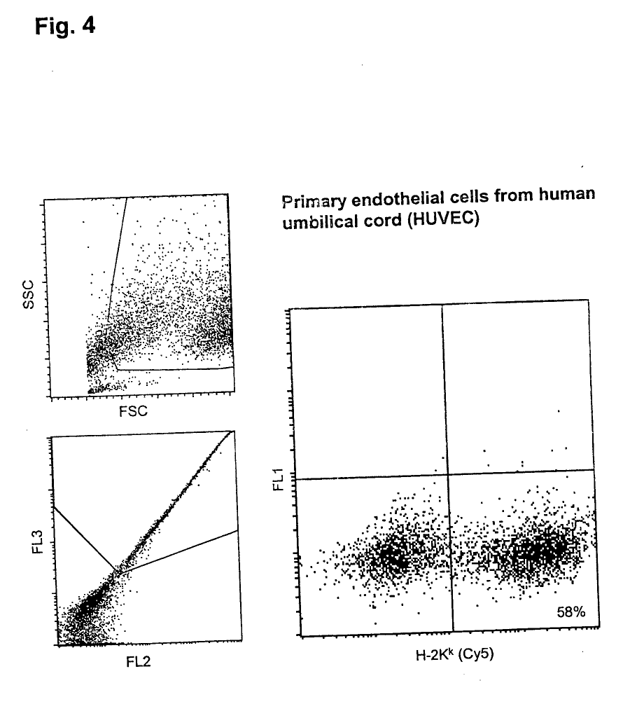

As shown in FIG. 4, 58% of the cells express the transfected DNA with a mortality of 32%. If DNA would have reached the cytoplasm by transfection in 100% of the cells having a division period of 24 h, and if no regeneration period after the transfection was considered, DNA could have reached the nucleus by disintegration of the nuclear envelope in max. 16% of the cells.

### Example 5

Electrical Nuclear Transport in a Cell Line (HeLa)

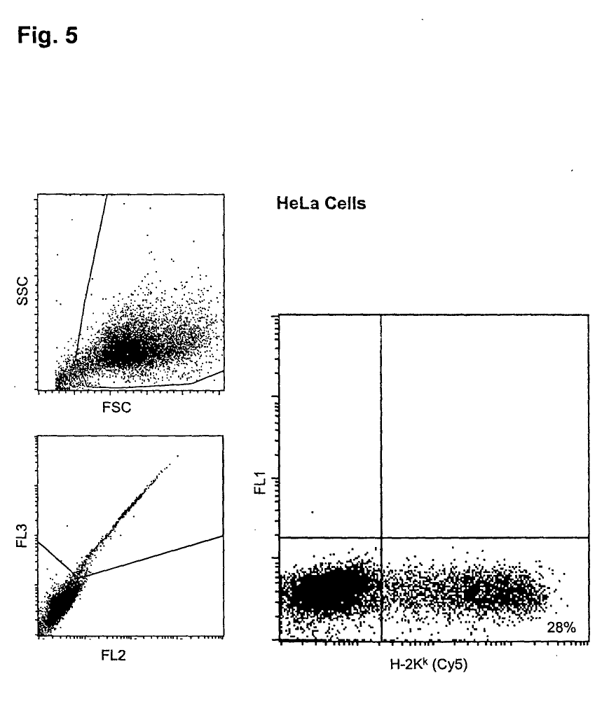

As described in example 4, HeLa cells were transfected by a 100 μs pulse of 4 kV/cm and analyzed after 3 hours. As shown in FIG. 5, 28% of the cells express the transfected DNA, and the mortality was 5.5%. If DNA would have reached the cytoplasm by transfection in 100% of the cells having a division period of 24 h, and if no regeneration period after the transfection was considered, DNA could have reached the nucleus by disintegration of the nuclear envelope in max. 12.5% of the cells after 3 h.

### Example 6

Transfection Efficiency of Various Unstimulated Primary Cells and Cell Lines

1×10⁶ PBMC, other primary cells or cell line cells were transfected according to the procedure described in example 1 with various expression vectors, and with the settings described in example 1. In the subsequently performed flow cytometric analysis (FACScan), the various subpopulations were identified by specific antibodies. In the following table 1, the mean transfection efficiencies are listed. For the analysis of the transfection of CD34⁺ precursor cells, 2.5-5×10⁶ PBMC were transfected. A first analysis of the transfection efficiencies was performed after 3.5 h, since during this time span no cells or only a few cells in cell lines have undergone division. Values marked with an asterix (*) were also determined 3 days after transfection, and were as high as the 24 h values.

### Example 7

Electrical Nuclear Transport of HPV18-E2 Protein in HeLa Cells

The electrical nuclear transport of proteins can be demonstrated for example by the concomitant transfection of transcription activator proteins and reporter constructs, the expression of which may be turned on by the binding of the activator molecules in the nucleus. Thus, HeLa cells were transfected with the vector pC18Sp1luc, a plasmid containing four binding sites for the papilloma virus transcription activator HPV18-E2 upstream of the promotor sequence as well as a luminescence reporter sequence, and with purified HPV18-E2 protein. At room temperature 1×10⁶ cells were transferred to a cuvette together with 200 ng vector DNA and 8 ng protein in buffer, and transfected with a 100 μs pulse of 4 kV/cm. After 6 h incubation at 37° C. and 5% CO₂ the cells were analyzed by measuring the relative luminescence activity.

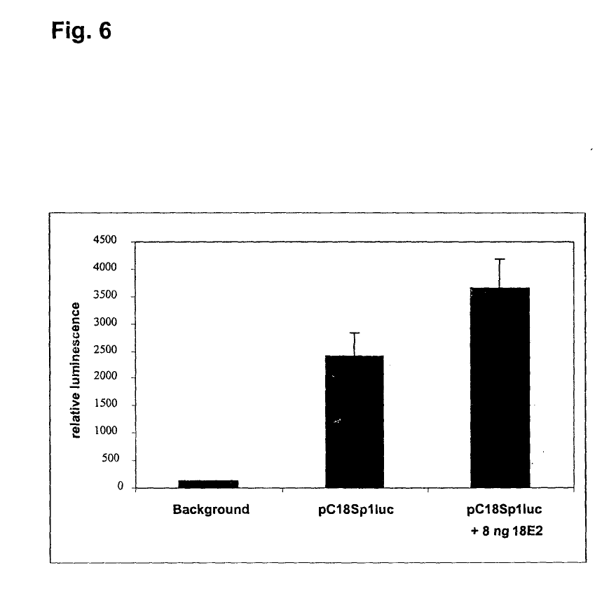

FIG. 6 shows the cotransfection of the vector DNA with the protein, the preparation without protein representing the control value. After cotransfection with 8 ng protein, a clear increase of the luminescence activity was observed compared to the controls, demonstrating that the transcription activator has reached the nucleus, and has resulted in an expression of the reporter sequence. Therefore, the method according to the invention allows the introduction of proteins into the nucleus of eukaryotic cells too.

### Example 8

Electrical Nuclear Transport of Antibodies

An electrical nuclear transport of antibodies may be obtained for example by the following setup. 1×10⁶ HeLa cells were transfected with an antibody directed against the nucleus-specific protein complex ND10 in 100 μl buffer solution at room temperature with a 10 μs pulse of 4 kV/cm, followed without interruption by a current flow of 5 A and 10 ms. Immediately after delivery of the pulse, the cells were rinsed out of the cuvette with 400 μl cell culture medium without serum, and were incubated for 10 min at 37° C. The cells were plated in 37° C. warm cell culture medium (with serum), and were incubated for 5 h at 37° C. and 5% CO₂. Then they were fixed in formaldehyde/glutaraldehyde, permeabilized with Triton X100, incubated with a FITC-labeled antibody specific for the anti-ND10 antibody, and analyzed by fluorescence microscopy.

### Example 9

Electrical Nuclear Transport of DNA-Protein Complexes in CHO Cells

The electrical nuclear transport of DNA-protein complexes may be shown for example by the repression of expression of transfected reporter plasmid-repressor complexes. For this purpose, CHO cells were transfected with a vector (pSpe(LacO)₁—H2K^{k}) having a lac-operator sequence between the promotor and the H2K^{k} marker sequence to which lac repressor molecules have been bound. Concomitantly the cells were contransfected with the vector pMACS4.1 coding for human CD4 and not containing a lac-operator sequence, so that lac repressor molecules cannot bind to it specifically. 1×10⁶ cells were incubated with 1 μg H2K^{k} expression vector DNA with lacO sequence and 1 μg CD4 expression vector DNA without lacO sequence in buffer at room temperature with 200 ng lac repressor protein for 30 min, transferred to a cuvette, and transfected with a pulse of 5 kV/cm for 70 μs, followed by a current flow of 2.2 A for 60 ms. After incubation for 5.5 h at 37° C. and 5% CO₂, the cells were trypsinized, stained and analyzed by flow cytometry for expression of the respective markers. H2K^{k} expression was analyzed by incubation with Cy5 coupled anti-H-2K^{k} antibody, and CD4 expression was analyzed by incubation with phycoerythrin (PE) coupled anti-CD4 antibody.

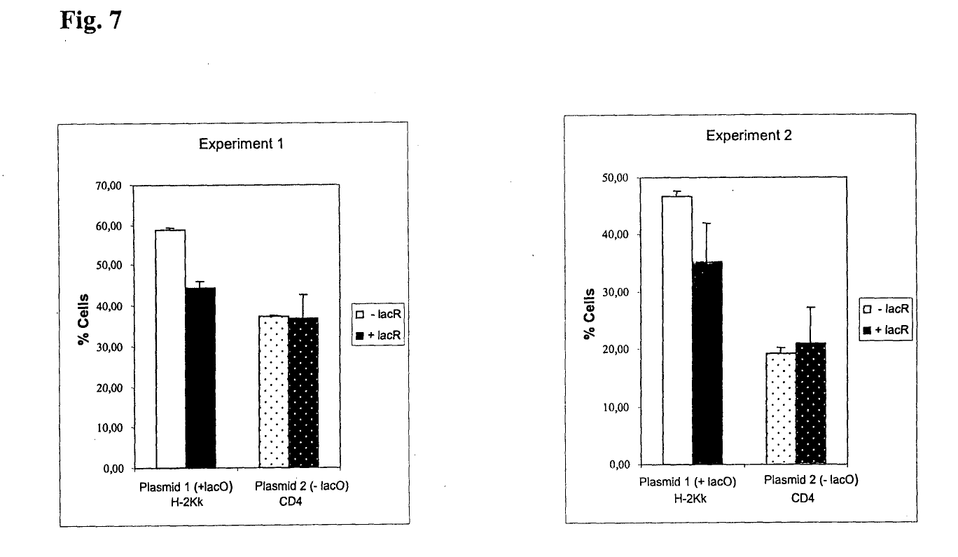

The results of two experiments shown in FIG. 7 demonstrate the expression of the marker sequence after transfection of the DNA-protein complexes with and without bound lac repressor. With specifically bound lac repressor (H2K^{k} expression vector with lacO sequence, plasmid 1), a clear repression of the H2K^{k} expression compared to the control without lac repressor can be seen, whereas without specific lac repressor binding (CD4 expression vector without lacO-sequence, plasmid 2) no repression of the CD4 expression occurred. This shows that the DNA-protein complex has reached the nucleus and that the repressor protein has resulted in a suppression of the expression of the marker sequence. Therefore, the method according to the invention also allows the introduction of DNA-protein complexes into the nucleus of eukaryotic cells.

### Example 10

Electrical Nuclear Transport of Peptide-DNA-Complexes

The nuclear transport of peptide-DNA complexes may be demonstrated for example by a repression of the expression of a reporter plasmid by binding of PNA (peptide nucleic acid) to a PNA-binding sequence between promotor and reporter cassette of a reporter plasmid prior to transfection. 1 μg H-2K^{k} expression vector were incubated in 10 mM Tris, 1 mM EDTA with 25 μM PNA peptide (low concentration), or 50 μM PNA peptide (high concentration) for 15 min at 65° C. The expression vector was used in two variations, with and without specific PNA-binding sequence, also, unspecific PNA peptides (peptide-1) and specifically binding PNA peptides (peptide 2) were used. Specific PNA binding resulted in the labelling of a restriction site, and was verified by respective restriction analysis. The used PNA peptides had the following DNA binding sequence: NH₂-CCTTTCTCCCTTC-peptide (peptide 1) or NH₂-CTCTTCCTTTTTC-peptide (peptide 2). The mere peptide portion had the following sequence: NH₂-GKPTADDQHSTPPKKKRKVED-COOH. For transfection of K562 cells, a peptide portion with the following sequence was used: NH₂-GKPSSDDEATADSQHSTPPKKKERKVED-COOH. After the incubation, the complexes were transfected by a pulse of 5 kV/cm for 70 μs, followed by a current flow of 2.2 A for 40 ms in CHO cells, and by a pulse of 5 kV/cm for 100 μs, followed by a current of 5 A for 10 ms in K562 cells. After incubation for 4 h at 37° C. and 5% CO₂, the cells were stained with CY5 coupled anti-H-2K^{k} antibody, and were analyzed for H-2K^{k} expression by flow cytometry.

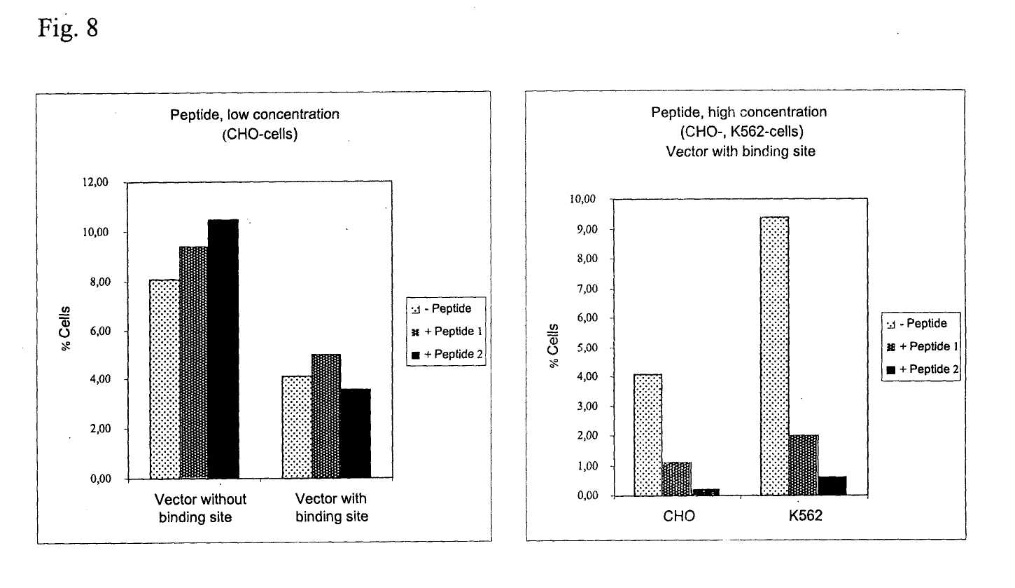

FIG. 8 shows the effect of specific binding and unspecific interaction of PNA peptide with vector DNA on the expression of a reporter construct, and therewith the peptide-DNA complex having reached the nucleus. Therefore, the method according to the invention also allows the introduction of peptide-DNA complexes into the nucleus of eukaryotic cells.

TABLE 1

| | | % + after | % + after |
|---|---|---|---|
| Cell type | Conditions | 3.5 h | 24 h |
| PBMC | | | |
| CD4⁺ (T_{H} cells) | 1000 V 40 μs/2.2 A | 30-60 | 35-70* |
| | 30 ms | | |
| CD8⁺ (T_{c} cells) | 1000 V 70 μs/2.2 A | 20-70 | 35-75* |
| | 10 ms | | |
| CD14⁺ (monocytes) | 1000 V 70 μs/1.0 A | 30-50 | 10-20 |
| | 1 ms | | |
| CD19⁺ (B cells) | 800 V 70 μs/2.0 A | 10-40 | 10-50* |
| | 30 ms | | |
| CD34⁺ (stem cells) | 1000 V 70 μs/2.2 A | 40-60 | 40-50 |
| | 10 ms | | |
| Umbilical cord | | | |
| HUVEC | 800 V 70 μs/2.0 A | 60-70 | 80-90 |
| | 10 ms | | |
| Embryonic chicken | | | |
| cells | | | |
| Neurons | 800 V 40 μs/0.1 A | 30 | 50 |
| | 10 ms | | |
| Fibroblasts | 1000 V 50 μs/0.1 A | 30 | 60 |
| | 1 ms | | |
| Cell lines | | | |
| HeLa | 1000 V 40 μs/2.2 A | >30 | n.d. |
| | 1 ms | | |
| CHO | 1000 V 50 μs/1.6 A | 20-30 | n.d |
| | 10 ms | | |

References

Auer, D., Brandner, G., Bodemer, W. (1976), Dielectric breakdown of the red blood cell membrane and uptake of SV40 DNA and mammalian RNA. Naturwissenschaften, 63: 391

Bertling, W., Hunger-Bertling, K., Cline, M. J. (1987), Intranuclear uptake and persistence of biologically active DNA after electroporation of mammalian cells. J Biochem Biophys Methods July; 14(4):223-32

Lurquin, P. F. (1997), Gene transfer by electroporation. Mol Biotechnol February; 7(1):5-35

Marechal, V., Dehee, A., Chikhi-Brachet, R., Piolot, T., Coppey-Moisan, M., Nicolas, J. -C. (1999), Mapping EBNA-1 domains involved in binding to metaphase chromosomes. J. Virol. 73:4385-4392

Neumann, E., Rosenheck, K. (1972), Permeability changes induced by electric impulses in vesicular membranes. J. Membrane Biol. 10: 279-290

Neumann, E., Schaefer-Ridder, M., Wang, Y., Hofschneider, P. H. (1982), Gene transfer into mouse lyoma cells by electroporation in high electric fields. EMBO J;1(7):841-5

Potter, H., Weir, L., Leder, P. (1984), Enhancer-dependent expression of human kappa immunoglobulin genes introduced into mouse pre-B lymphocytes by electroporation. Proc Natl Acad Sci USA November; 81(22):7161-5

Zimmermann, U., Scheurich, P., Pilwat, G., Benz, R. (1981), Cells with manipulated functions: new perspectives for cell biology, medicine and technology. Angew. Chem. Int. Ed. Engl. 20: 325-344

Cited Patents

U.S. Pat. No. 4,750,100, Ragsdale, C. W. (1988), Transfection high voltage controller

U.S. Pat. No. 5,869,326, Hofmann, G. A. (1999), Electroporation employing user-configured pulsing scheme

U.S. Pat. No. 6,008,038/EP 0 866123 A1, Kroger, W., Jagdhuber, B., Ricklefs, H -J. (1999), Method and a circuit arrangement for the electropermeation of living cells

1) A method for introducing biologically active molecules into eukaryotic cells using electric current, the introduction into the nucleus being achieved by a pulse having a field strength of min. 2 kV/cm and a duration of at least 10 μs,
characterized in that
the biologically active molecules are introduced into the nucleus by a pulse having a field strength of max. 10 kV/cm and a current of at least 1 A.

2) The Method according to claim 1, wherein the field strength of the pulse is 3-8 kV/cm.

3) The method according to claims 1 or 2, wherein the pulse has a duration of max. 200 μs.

4) The method according to any of the claims 1 to 3, wherein the pulse is followed without interruption by a current flow of 1 A to max. 2.5 A with a duration of 1 ms to max. 50 ms.

5) The method according to any of the claims 1 to 4, wherein the biologically active molecule comprises a nucleic acid, or at least includes a nucleic acid portion.

6) The method according to claim 5, wherein the nucleic acid is present in complex or in association with peptides, proteins, polysaccharides, lipids, or combinations or derivatives of these molecules.

7) The method according to claim 6, wherein the molecules complexed or associated with the nucleic acid are used for the integration of the transferred nucleic acid in the genome of the cell, the intranuclear localization or retention, the association with the chromatin, or the regulation of the expression.

8) The method according to claim 6, wherein the molecules complexed with the nucleic acid which are used for the integration of the transferred nucleic acid in the genome of the cell are selected from the group comprising retroviral integrases, prokaryotic transposases, eukaryotic transposases, sequence-specific recombinases, topoisomerases, E. coli recA, E. coli recE, E. coli recT, phage λ red α, phage λ red β and phage λ terminase.

9) The method according to claim 6, wherein the molecules complexed or associated with the nucleic acid which are used for the intranuclear retention or the association with chromatin comprise domains of the EBV protein EBNA-1.

10) The method according to any of the claims 1-9, wherein the eukaryotic cells are resting non-dividing cells.

11) The method according to any of the claims 1-9, wherein the eukaryotic cells are dividing cells.

12) The method according to any of the claims 1-9, wherein the eukaryotic cells are primary cells.

13) The method according to claim 12, wherein the eukaryotic cells are peripheral human blood cells.

14) The method according to claim 12, wherein the eukaryotic cells are pluripotent precursor cells of human blood.

15) The method according to claim 12, wherein the eukaryotic cells are embryonic cells from human, rat, mouse, and chicken neurons.

16) The method according to claim 12, wherein the eukaryotic cells are human bone marrow cells.

17) The method according to claims 1-16, wherein the transfected eukaryotic cells are used for diagnostic methods.

18) A use of eukaryotic cells transfected according to any of the claims 1-17 for preparating a drug for ex vivo gene therapy.
